SEQUENCE LISTING (2 sequences)

SEQ ID NO: 1 - DNA, 739 bp, Artificial Sequence
  hu4D5-8
ccatggatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaaggcggtggtggcagcggtggcggtggcagcggcggtggcggtagcgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcggcggccgc

SEQ ID NO: 2 - protein, 256 aa, Artificial Sequence
  hu4D5-8scFv
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAALEHHHHHHGGC

(19) United States
(12) Patent Application Publication
(10) Pub. No.: US 2013/0315837 A1
(43) Pub. Date: Nov. 28, 2013

(54) DYE-CONTAINING NANOPARTICLE FOR PHOTOACOUSTIC CONTRAST AGENT

(71) Applicant: Canon Kabushiki Kaisha, Tokyo (JP)

(72) Inventors: Fumiko Tomatsu, Yokohama-shi (JP); Tatsuki Fukui, Yokohama-shi (JP); Kengo Kanazaki, Kyoto-shi (JP); Satoshi Yuasa, Yokohama-shi (JP); Mayuko Kishi, Machida-shi (JP); Daisuke Sasaguri, Yokohama-shi (JP)

(73) Assignee: Canon Kabushiki Kaisha, Tokyo (JP)

(21) Appl. No.: 13/960,377

(22) Filed: Aug. 6, 2013

Related U.S. Application Data
(63) Continuation of application No. PCT/JP2013/000995, filed on Feb. 21, 2013.

(30) Foreign Application Priority Data
Feb. 23, 2012 (JP) 2012-038036
Nov. 30, 2012 (JP) 2012-263003

(51) Int. Cl. A61K 49/22

(52) U.S. Cl. CPC A61K 49/22
USPC 424/9.6

(57) ABSTRACT

An object of the present invention is to increase the dye content in nanoparticles and to improve the signal intensity per particle. According to the nanoparticle including at least a silicon naphthalocyanine or a derivative thereof and a surfactant, wherein the proportion of the silicon naphthalocyanine or the derivative thereof is 70% or more by weight in relation to the other component of the particle exclusive of the surfactant, the dye content in the nanoparticles can be increased and the signal intensity per particle can be improved without weakening the signal intensity per dye molecule (light absorptivity)

### CROSS-REFERENCE TO RELATED APPLICATIONS

This application is a continuation of International Application No. PCT/JP2013/000995, filed Feb. 21, 2013, which claims the benefit of Japanese Patent Applications No. 2012-038036, filed Feb. 23, 2012 and No. 2012-263003, filed Nov. 30, 2012.

### BACKGROUND OF THE INVENTION

1. Field of the Invention

The present invention relates to a dye-containing nanoparticle for a photoacoustic contrast agent.

2. Description of the Related Art

Recently, as a method for noninvasively visualizing intra-organism information, a photoacoustic imaging method and a fluorescence imaging method have been attracting attention.

In a measurement using the photoacoustic imaging method, an object to be measured is irradiated with light, and an intensity and a time of occurrence of the photoacoustic signal emitted by a substance (optical absorber) absorbing the light in the interior of the object are measured, and herewith the distribution of the substance in the interior of the object to be measured can be visualized by computational operation.

In a measurement using the fluorescence imaging method, an object to be measured is irradiated with light, and a fluorescence emitted by an optical absorber in the interior of the object is measured, and herewith the distribution of the substance in the interior of the object can be visualized.

Here, as the optical absorber, a substance absorbing light and emitting acoustic wave or fluorescence inside an organism can be suitably used. For example, by using a blood vessel, a malignant tumor or the like in the inside of human body as an optical absorber, the acoustic wave emitted from the optical absorber can be measured. Otherwise, for example, a dye absorbing light in the near-infrared region is administered into a body, and thus can be used as a contrast agent. Since the light in the near-infrared region is small in the effect on the human body when the human body is irradiated with the light concerned and it has a high degree of translucency through organisms, a dye which can absorb the light concerned can be suitably used as the contrast agent in the photoacoustic imaging method and the contrast agent in the fluorescence imaging method. The dye as referred to in present DESCRIPTION is defined as a compound capable of absorbing light falling within the wavelength range from 600 nm to 1300 nm.

In such a contrast agent, for the purpose of effectively enhance the signal intensity (intensity of acoustic wave or fluorescence), desirably, by accumulating a dye, for example, inside a particle, a micelle, a polymer micelle or a liposome (generically referred to as a particle or the like), the dye density is increased and thus the absorption efficiency of the irradiation energy is increased. As such a particle or the like, for example, Japanese Patent Application Laid-Open No. 2010-083860 discloses a polymer nanoparticle obtained by a nanoemulsion method, containing a silicon naphthalocyanine and having its particle surface protected with a surfactant. Also, Photochemistry and Photobiology, 1996, 63(1), 132-140 discloses a silicon naphthalocyanine-containing nanoparticle obtained by dissolving a silicon naphthalocyanine in tetrahydrofuran (THF), a hydrophilic solvent and by using Tween 80 as a surfactant.

### SUMMARY OF THE INVENTION

In Japanese Patent Application Laid-Open No. 2010-083860, since the silicon naphthalocyanine and a polymer are contained inside a particle, there is a problem such that the dye content in the inside of a particle cannot be increased, and the improvement of the signal intensity per a particle is hardly achieved.

Also, in Photochemistry and Photobiology, 1996, 63(1), 132-140, particles are prepared by dissolving the silicon naphthalocyanine in THF, a hydrophilic solvent, and a purification by filtration with a 0.45 μm filter is performed. However, such a technique causes a problem such that a large number of large particles would be obtained.

The present invention has been achieved in view of such problems as described above, and an object of the present invention is to provide a particle capable of obtaining a signal having a high intensity by light irradiation. Another object of the present invention is to provide a particle having a size appropriate for a contrast agent.

The nanoparticle according to the present invention contains a silicon naphthalocyanine or a derivative thereof, the surface of the particle is protected with a surfactant, and additionally, the proportion of the silicon naphthalocyanine or the derivative thereof in relation to the other component of the particle exclusive of the surfactant is 70% or more by weight.

According to the present invention, a nanoparticle capable of producing a signal having a higher intensity when used as a contrast agent can be provided.

Further features of the present invention will become apparent from the following description of exemplary embodiments with reference to the attached drawings.

### BRIEF DESCRIPTION OF THE DRAWINGS

FIG. 1A is the photoacoustic image of a tumor site of a tumor-bearing mouse, prior to the administration of NP9 to the mouse.

FIG. 1B is the photoacoustic image of the tumor site of the tumor-bearing mouse, after the passage of 24 hours from the administration of 26 nmol of NP9 as a dye amount to the tumor-bearing mouse.

FIG. 2 is a graph showing the photoacoustic signal intensity ratio (at 24 hours after the administration/prior to the administration) of the tumor site in relation to administration of NP9 to a tumor-bearing mouse.

### DESCRIPTION OF THE EMBODIMENTS

### Nanoparticle

The nanoparticle according to the present embodiment includes a silicon naphthalocyanine or a derivative thereof, and a surfactant bonded to its particle surface. The nanoparticle in the present embodiment is defined as a particle having a particle size of the order of nm (nanometers), namely, less than 1000 nm. However, for example, in the actual use of the nanoparticle as the below described contrast agent, even in the case where the particles having a particle size of 1000 nm or more are included in a set of the nanoparticles, the set of the nanoparticles is included in the definition of the nanoparticle of the present invention if its average particle size is less than 1000 nm.

(Content of Silicon Naphthalocyanine or Derivative Thereof inside Particles)

In the nanoparticle according to the present embodiment, the proportion of silicon naphthalocyanine or the derivative thereof in relation to the other component of the particle exclusive of the surfactant is 70% or more and less than 100% by weight. The proportion of the silicon naphthalocyanine or the derivative thereof in relation to the other component of the particle exclusive of the surfactant is preferably 80% or more and less than 100% by weight, more preferably 90% or more and less than 100% by weight, and particularly preferably 95% or more by weight. By increasing the proportion of the dye included inside the particles, the absorption efficiency of the irradiation energy is increased when the particles are used as a contrast agent, and even by using a smaller number of particles, a signal having a higher intensity can be obtained. When the nanoparticles according to the present embodiment are used in the photoacoustic imaging method, the accumulation of the dye inside the particles causes the quenching of the fluorescence so as to prevent the irradiation energy from being used for fluorescence emission, and allows the irradiation energy to be transformed into a larger amount of thermal energy. Consequently, the acoustic signal can be obtained more efficiently.

In the present embodiment, the dye can be accumulated in a high concentration inside the particles while the effect of the decrease of the light absorption amount per dye molecule is being suppressed to be low or the light absorption amount is absolutely not decreased.

(Particle Size)

The particle size of the nanoparticles according to the present embodiment is preferably 5 nm or more and less than 1000 nm. When the particle size is less than 1000 nm, due to the Enhanced Permeability and Retention (EPR) effect, the nanoparticles can be accumulated in the tumor sites in a larger number than in the normal sites. By detecting the accumulated nanoparticles, for example, with photoacoustic tomography, the imaging of the tumor sites can be specifically performed. The particle size of the nanoparticles is more preferably 5 nm or more and 500 nm or less and furthermore preferably 5 nm or more and 200 nm or less. This is because when the particle size of the nanoparticles is 200 nm or less, the nanoparticles are hardly engulfed by the macrophage in blood, and the retention of the nanoparticles in blood is considered to become high.

When the particle size of the nanoparticles is referred to in the present embodiment, the particle size is the hydrodynamic diameter measured by the dynamic light scattering (DLS) method with a dynamic light scattering analyzer (DLS-8000, manufactured by Otsuka Electronics Co., Ltd.).

(Silicon Naphthalocyanine or Derivative Thereof)

The silicon naphthalocyanine or the derivative thereof according to the present embodiment may be any compound having a naphthalocyanine skeleton and a silicon compound in the center. Since the naphthalocyanine skeleton is hydrophobic, silicon naphthalocyanine having the naphthalocyanine skeleton or the derivative thereof tends to gather to form an aggregation of the molecules of the compound concerned through hydrophobic interaction. The aggregation of the molecules of the silicon naphthalocyanine or the derivative thereof becomes further higher in hydrophobicity. Consequently, when the nanoparticles according to the present embodiment are placed in an aqueous solution like serum, the silicon naphthalocyanine or the derivative thereof becomes difficult to leak outside the particles.

In the present embodiment, the structure of silicon naphthalocyanine or the derivative thereof is represented by the chemical formula 1,

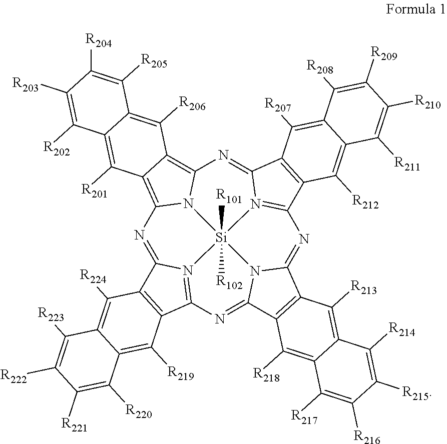

In the formula, R₂₀₁, R₂₀₂, R₂₀₃, R₂₀₄, R₂₀₅, R₂₀₆, R₂₀₇, R₂₀₈, R₂₀₉, R₂₁₀, R₂₁₁, R₂₁₂, R₂₁₃, R₂₁₄, R₂₁₅, R₂₁₆, R₂₁₇, R₂₁₈, R₂₁₉, R₂₂₀, R₂₂₁, R₂₂₂, R₂₂₃ and R₂₂₄ may each be the same or different, and each represent a hydrogen atom, a halogen atom, an acetoxy group, an amino group, a nitro group, a cyano group or an alkyl group having 1 to 18 carbon atoms or aromatic group which is unsubstituted or substituted with one or a plurality of the functional groups selected from a halogen atom, an acetoxy group, an amino group, a nitro group, a cyano group and an alkyl group having 1 to 18 carbon atoms.

R₁₀₁ and R₁₀₂ may each be the same or different, and each represent —OH, —OR₁₁, —OCOR₁₂, —OSi(—R₁₃)(—R₁₄)(—R₁₅), a halogen atom, an acetoxy group, an amino group, a nitro group, a cyano group or an alkyl group having 1 to 18 carbon atoms or aromatic group which is unsubstituted or substituted with one or a plurality of the functional groups selected from a halogen atom, an acetoxy group, an amino group, a nitro group, a cyano group and an alkyl group having 1 to 18 carbon atoms.

Here, R₁₁, R₁₂, R₁₃, R₁₄ and R₁₅ may each be the same or different, each represent a group unsubstituted or substituted with one or a plurality of the functional groups selected from a halogen atom, an acetoxy group, an amino group, a nitro group, a cyano group and an alkyl group having 1 to 18 carbon atoms.

Examples of the silicon naphthalocyanine may include: Silicon 2,3-naphthalocyanine dihydroxide, Silicon 2,3-naphthalocyanine dioctyloxide, Silicon 2,3-naphthalocyanine dichloride, Bis(di-isobutyl octadecylsiloxy)silicon 2,3-naphthalocyanine, Silicon 2,3-naphthalocyanine bis(trihexylsilyloxide) (hereinafter, also abbreviated as the compound 1, in some cases); particularly preferable among these is silicon 2,3-naphthalocyanine bis(trihexylsilyloxide).

The silicon naphthalocyanine or the derivative thereof has an absorption in the near-infrared region from 600 nm to 900 nm, which is excellent in the translucency through organisms. The nanoparticles according to the present embodiment contain the silicon naphthalocyanine or the derivative thereof, and hence can absorb the light of the wavelengths in the near-infrared region (the near-infrared region from 600 nm to 900 nm) being safe in the irradiation of orgasms therewith and having a relatively high translucency through organisms.

(Surfactant)

As the surfactant in the present embodiment, for example, a non-ionic surfactant, an anionic surfactant, a cationic surfactant, a polymeric surfactant, a phospholipid or a polysaccharide can be used. These surfactants may be used each alone or in combinations of two or more thereof.

Examples of the non-ionic surfactant used as the surfactant in the present embodiment include: polyoxyethylene sorbitan-based fatty acid esters (for example, a compound represented by the chemical formula 2), Brij (registered trademark) 35, Brij (registered trademark) 58, Brij (registered trademark) 76, Brij (registered trademark) 98, Triton (registered trademark) X-100, Triton (registered trademark) X-114, Triton (registered trademark) X-305, Triton (registered trademark) N-101, Nonidet (registered trademark) P-40, IGEPAL (registered trademark) CO530, IGEPAL (registered trademark) CO630, IGEPAL (registered trademark) CO720 and IGEPAL (registered trademark) CO730.

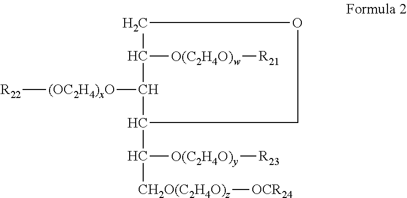

In the chemical formula 2, R₂₁ to R₂₄ are each independently selected from —H and —OCR′. The R′ is a saturated or unsaturated alkyl group having 1 to 18 carbon atoms. In the chemical formula 2, w, x, y and z are integers giving the sum of w, x, y and z to be 10 to 30.

Examples of the polyoxyethylene sorbitan-based fatty acid esters represented by the chemical formula 2 may include Tween (registered trademark) 20, Tween (registered trademark) 40, Tween (registered trademark) 60, Tween (registered trademark) 80 and Tween (registered trademark) 85. Among these, Tween (registered trademark) 20 and Tween (registered trademark) 80 are particularly preferable.

Examples of the anionic surfactant used as the surfactant in the present embodiment may include: dodecylsulfuric acid, dodecylbenzenesulfonate, decylbenzenesulfonate, undecylbenzenesulfonate, tridecylbenzenesulfonate and nonylbenzenesulfonate, and sodium, potassium and ammonium salts of these; and sodium, potassium and ammonium salts of lauric acid, myristic acid, palmitic acid, stearic acid and oleic acid.

Examples of the cationic surfactant used as the surfactant in the present embodiment may include cetyltrimethylammonium bromide, hexadecylpyridinium chloride, dodecyltrimethylammonium chloride and hexadecyltrimethylammonium chloride.

Examples of the polymeric surfactant used as the surfactant in the present embodiment may include polyvinyl alcohol, polyoxyethylene-polyoxypropylene block copolymer and gelatin. Examples of the polyoxyethylene-polyoxypropylene block copolymer include the compounds represented by the chemical formula 3. In the chemical formula 3, x and z are each independently an integer of 70 or more and 110 or less and can be an integer of 75 or more and 106 or less. In the chemical formula 3, y is an integer of 20 or more and 80 or less and can be an integer of 30 or more and 70 or less. Examples of the block copolymer in which x and z are each 75 and y is 30 in the chemical formula 3 may include Pluronic (registered trademark) F68, and examples of the block copolymer in which x and z are each 106 and y is 70 in the chemical formula 3 may include Pluronic (registered trademark) F127.

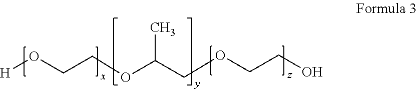

The phospholipid used as the surfactant in the present embodiment can be a phosphatidyl-based phospholipid, having any functional group of a hydroxyl group, a methoxy group, an amino group, a carboxyl group, an N-hydroxysuccinimide group and a maleimide group. The phospholipid used as the surfactant may be a phospholipid including a PEG (Polyethylene glycol) chain.

Examples of the phospholipid used as the surfactant including a functional group such as a hydroxyl group, a methoxy group, an amino group, an N-hydroxysuccinimide group or a maleimide group, and also including a PEG chain may include: 1,2-distearoyl-sn-glycero-3-phosphoethanolamine-N-[carboxy(polyethylene glycol)] (DSPE-PEG-OH), Poly(oxy-1,2-ethanediyl), α-[7-hydroxy-7-oxido-13-oxo-10-[(1-oxooctadecyl)oxy]-6,8,12-trioxa-3-aza-7-phosphatriacont-1-yl]-ω-methoxy-(DSPE-PEG-OMe), N-(aminopropyl polyethyleneglycol)-carbamyl distearoylphosphatidyl-ethanolamine (DSPE-PEG-NH2), 3-(N-succinimidyloxyglutaryl)aminopropyl polyethyleneglycol-carbamyl distearoylphosphatidyl-ethanolamine (DSPE-PEG-NHS), N-(3-maleimide-1-oxopropyl)aminopropyl polyethyleneglycol-carbamyl distearoylphosphatidyl-ethanolamine (DSPE-PEG-MAL), SUNBRIGHT (registered trademark) DSPE-020-PA, SUNBRIGHT (registered trademark) DSPE-020-CN, SUNBRIGHT (registered trademark) DSPE-050-CN, Methoxyl PEG DSPE, Mw10000, Methoxyl PEG DSPE, and Mw 20000.

Examples of the polysaccharide used as the surfactant in the present embodiment may include dextran and heparin.

(Method for Producing Nanoparticle)

As the method for producing a particle of the present invention, heretofore known methods can be used. Examples of such a method include a nanoemulsion method and a nanoprecipitation method.

Examples of the solvent used in the present production method may include: hydrocarbons such as hexane, cyclohexane and heptane; ketones such as acetone and methyl ethyl ketone; ethers such as diethyl ether and tetrahydrofuran; halogenated hydrocarbons such as dichloromethane, chloroform, carbon tetrachloride, dichloroethane and trichloroethane; aromatic hydrocarbons such as benzene and toluene; esters such as ethyl acetate and butyl acetate; polar aprotic solvents such as N,N-dimethylformamide and dimethyl sulfoxide; and pyridine derivatives. These solvents may be used each alone or as optional mixtures thereof.

In the nanoemulsion method, an emulsion can be prepared by a heretofore known emulsification technique. Examples of the heretofore known method include: an intermittent shaking method, a stirring method using a mixer such as a propeller type stirrer or a turbine type stirrer; a colloid mill method, a homogenizer method and an ultrasonic wave irradiation method. These methods can be used each alone or in combinations of a plurality thereof. The emulsion may be prepared by a single-stage emulsification or a multistage emulsification. The emulsification technique is not limited to the foregoing techniques as long as the objects of the present invention can be achieved.

In the nanoprecipitation method, particles can be prepared by a heretofore known method in which an organic solvent dispersion is mixed and stirred in a surfactant dispersed aqueous solution, or a method in which a surfactant dispersed aqueous solution is mixed and stirred in an organic solvent dispersion.

(Organic Solvent Dispersion Dissolving Material Including Silicon Naphthalocyanine or Derivative Thereof)

In the nanoemulsion method, the weight ratio between the surfactant dispersed aqueous solution used and the organic solvent dispersion used is not particularly limited as long as an oil-in-water (O/W) type emulsion can be formed. The weight ratio between the organic solvent dispersion and the aqueous solution can be within a range from 1:2 to 1:1000.

In the nanoprecipitation method, the weight ratio between the surfactant dispersed aqueous solution used and the organic solvent dispersion used is not particularly limited as long as the particles can be collected. The weight ratio between the organic solvent dispersion and the aqueous solution can be within a range from 1:1 to 1:1000.

(Material Concentration in Organic Solvent Dispersion Dissolving Material Including Silicon Naphthalocyanine or Derivative Thereof)

The concentration of silicon naphthalocyanine or the derivative thereof in the organic solvent dispersion is not particularly limited as long as the concentration falls within the range in which each of these can be dissolved. The preferable concentration can be set at 0.0005 to 300 mg/ml.

(Distillation Off of Organic Solvent from Particle Dispersion)

The distillation off can be performed by any of the heretofore known methods; examples of the distillation off method may include a method of removal by heating and a method using a pressure-reducing device such as an evaporator.

In the nanoemulsion method, the heating temperature in the removal by heating is not particularly limited as long as the O/W type emulsion can be maintained; however, the preferable temperature is in a range from 0° C. to 80° C.

In the nanoprecipitation method, the heating temperature in the removal by heating is not particularly limited as long as the higher order aggregation to decrease the yield of the particles can be prevented; however, the preferable temperature is in a range from 0° C. to 80° C.

The distillation off is not limited to the foregoing techniques within a range where the objects of the present invention can be achieved.

(Purification of Particle Dispersion)

The purification of the prepared particle dispersion can be performed even by any of the heretofore known methods. For example such a purification method may including a size exclusion column chromatography method, an ultrafiltration method, a dialysis method and a centrifugal separation method can be used.

However, the purification method is not limited to the foregoing methods within a range where the objects of the present invention can be achieved.

(Particles)

The particles according to the present embodiment may have any shape as long as the particles are the particles including the hydrophobic metal phthalocyanine dye; the shape of the particles may be any of, for example, a spherical shape, an elliptical shape, a planar shape and a one-dimensional string shape. The size (particle size) of the particles according to the present embodiment is not particularly limited; however, the size of the particles can be 1 nm or more and 200 nm or less.

(Capture Molecule)

In the present embodiment, by immobilizing the capture molecules on the nanoparticles, the target-specific contrast agent can be made.

The capture molecule refers to, for example, a substance to be specifically bound to the target site such as a tumor, or a substance specifically bound to a substance present in the periphery of the target site, and it can be optionally selected from, for example, the chemical substances such as biomolecules and medicines. Specific examples of the capture molecule include antibodies, antibody fragments, enzymes, biologically active peptides, glycopeptides, sugar chains, lipids and molecular recognition compounds. These substances can be used each alone or in combinations of a plurality thereof.

By using the nanoparticles to which capture molecules are chemically bonded, a specific detection of the target site, and dynamics, localization and metabolism of the target substance can be performed.

(Immobilization of Capture Molecule)

The method for immobilizing the capture molecule on a nanoparticle depends on the type of the capture molecule to be used; however, any heretofore known method can be used as long as the method can chemically bond the capture molecule to the nanoparticle. For example, the method can be used in which by allowing a functional group possessed by a first surfactant or a second surfactant and a functional group of the capture molecule to react with each other, the capture molecule is chemically bonded to the nanoparticle.

For example, when the first surfactant or the second surfactant is a phosphatidyl-based phospholipid having an N-hydroxysuccinimide group, by allowing the capture molecule having an amino group to react with the surfactant, the capture molecule can be immobilized on the nanoparticle. After the immobilization of the capture molecule, the unreacted N-hydroxysuccinimide group of the surfactant can be deactivated by allowing the unreacted N-hydroxysuccinimide group to react, for example, with glycine, ethanolamine, or an oligoethylene glycol or polyethylene glycol having an amino group on the terminal thereof.

When the first surfactant or the second surfactant is a phosphatidyl-based phospholipid having a maleimide group, by allowing the maleimide group to react with a capture molecule having a thiol group, the capture molecule can be immobilized on the nanoparticle. After the immobilization of the capture molecule, the unreacted maleimide group of the surfactant can be deactivated by allowing the unreacted maleimide group to react, for example, with L-cysteine, mercaptoethanol, or oligoethylene glycol or polyethylene glycol having a thiol group on the terminal thereof.

When the first surfactant or the second surfactant is a phosphatidyl-based phospholipid having an amino group, by allowing the amino group to react with the amino group of the capture molecule by using glutaraldehyde, the capture molecule can be immobilized on the nanoparticle. After the immobilization of the capture molecule, the activity of the unreacted amino group of the surfactant can be blocked by allowing, for example, ethanolamine, or the oligoethylene glycol or the polyethylene glycol having an amino group on the terminal thereof to react with the unreacted amino group of the surfactant. Alternatively, the amino group of the surfactant is substituted with an N-hydroxysuccinimide group or a maleimide group, and then the capture molecule may be immobilized.

(Contrast Agent)

The contrast agent according to the present embodiment has the nanoparticles according to the present embodiment and the dispersion medium in which the nanoparticles are dispersed. The contrast agent according to the present embodiment may have, if necessary, pharmacologically acceptable additives, in addition to the nanoparticles according to the present embodiment.

The dispersion medium is a liquid substance for dispersing the particles according to the present embodiment, and examples of such a liquid substance include saline, distilled water for injection and a phosphate buffer. The contrast agent according to the present embodiment may be prepared beforehand by dispersing the nanoparticles according to the present embodiment in the dispersion medium, or the nanoparticles according to the present embodiment and the dispersion medium may be combined to form a kit, and the particles are dispersed in the dispersion medium just before the administration in the organism.

The nanoparticles according to the present embodiment hardly undergo the leakage of the silicon naphthalocyanine or the derivative thereof, and hence the silicon naphthalocyanine or the derivative thereof can be contained in the particles in a large amount. With the increase of the amount of the contained dye, the light absorption amount is increased, and hence the nanoparticles according to the present embodiment are suitable, as described below, for use in photoacoustic imaging or for use in fluorescence imaging. When a hydrophobic dye is contained in such a large amount that causes concentration quenching, the nanoparticles according to the present embodiment are further suitable for use in photoacoustic imaging.

(Imaging Method)

Referring now to the method for detecting the nanoparticles, according to the present embodiment, administered in an organism by using the photoacoustic imaging method, the method for detecting the nanoparticles according to the present embodiment includes the following steps. However, the imaging method according to the present embodiment may include additional steps other than the steps shown below.

(a) A step of administering the nanoparticles according to the present embodiment in an organism.

(b) A step of irradiating the organism with light and detecting photoacoustic signal emitted from inside of the organism.

In the step (a), the method for administering the nanoparticles according to the present embodiment in an organism is not particularly limited, and it can, for example, be oral administration or injection.

In the step (b), the light for irradiating the organism therewith can be a ray having a near-infrared wavelength falling within a range from 600 nm to 900 nm, such a ray being safe and exhibiting a high translucency through organisms when the organisms are irradiated with the ray. The light-generating apparatus and the acoustic signal-detecting apparatus are not particularly limited; various apparatuses can be used as such apparatuses.

The imaging method using the nanoparticles according to the present embodiment can image a site such as a tumor site by performing the steps (a) and (b).

Referring next to the method for detecting the complex according to the present embodiment administered in an organism by using the fluorescence imaging method is described, the method for detecting the nanoparticles according to the present embodiment includes the following steps. However, the imaging method according to the present embodiment may include additional steps other than the steps shown below.

(c) A step of administering the nanoparticles according to the present embodiment in an organism.

(d) A step of irradiating the organism with light and detecting fluorescence emitted from inside of the organism.

In the step (c), the method for administering the nanoparticles according to the present embodiment in an organism is not particularly limited, and it can, for example, be oral administration or injection.

In the step (d), the light for irradiating the organism therewith can be a ray having a near-infrared wavelength falling within a range from 600 nm to 900 nm, such a ray being safe and exhibiting a high translucency through organisms when the organisms are irradiated with the ray. The light-generating apparatus and the fluorescence detecting apparatus are not particularly limited; various apparatuses can be used as such apparatuses.

The imaging method using the nanoparticles according to the present embodiment can image a site such as a tumor by performing the steps (c) and (d).

When nanoparticles including the capture molecules are used in an organism, various target sites can be specifically detected by appropriately selecting the capture molecules. For example, when a substance specifically bound to a tumor is adopted as the capture molecule, a specific detection of the tumor can be performed. When a substance specifically bound to a biological substance such as a protein or an enzyme abounding in the periphery of a specific disease site is used as the capture molecule, the disease can be specifically detected. Even when the nanoparticles according to the present embodiment do not include the capture molecule, the tumor can be detected with the aid of the EPR effect.

(Method for Quantitatively Determining Dye Proportion)

The proportion of the dye in relation to the other component of the particle, exclusive of the surfactant, of the present invention can be determined, for example, by performing the following steps.

(1) A step of separating the nanoparticles from the fractions other than the nanoparticles.

(2) A step of analyzing the components of the nanoparticles.

In the step (1), the method for separating the nanoparticles according to the present embodiment from the fractions other than the particles is not particularly limited, and for example, can be based on centrifugal separation or ultrafiltration.

In the step (2), the method for analyzing the nanoparticles is not particularly limited, and examples of the possible methods include a method in which the particles are dissolved in a solvent and the quantitative determination of the components is performed by a separation analysis technique such as chromatography, and a method in which the particles are dissolved in a solvent and the quantitative determination of the known components is performed by an intrinsic component identification technique such as NMR. The solvent is not particularly limited, and for example, halogen-based solvents such as chloroform, and additionally, DMF and DMSO can be used.

### EXAMPLES

The specific reagents and the specific reaction conditions and so on used in Examples for the preparation of the nanoparticles of the present invention as described hereinafter can be appropriately altered, and such alterations are regarded as falling within the scope of the present invention. Accordingly, the following Examples are presented for the purpose of helping understand the present invention, but do not limit the scope of the present invention.

(Method for Measuring Photoacoustic Signal Intensity)

In the measurement of the photoacoustic signal intensity, a sample vessel placed in ultrapure water was irradiated with a pulse laser light, the intensity of the photoacoustic signal emitted from the sample in the vessel was detected by using a piezoelectric element, and the detected signal was amplified with a high speed preamplifier and acquired with a digital oscilloscope. The specific conditions are as follows. As the light source, a titanium sapphire laser (LT-2211-PC, manufactured by Lotis Ltd.) was used. The conditions were such that the wavelength was 780 nm, the energy density was about 10 to 20 mJ/cm², the pulse width was about 20 nanoseconds, and the frequency of the pulse repetition was 10 Hz. As the piezoelectric element for detecting the photoacoustic signal, a non-focusing type ultrasonic wave transducer (V303, manufactured by Panametrics-NDT Ltd.) having an element diameter of 1.27 cm and a central band of 1 MHz was used. The measurement vessel was a polystyrene cuvette, having an optical path length of 0.1 cm and a sample volume of about 200 μL. In a glass vessel filled with water, the measurement vessel and the piezoelectric element were immersed, and the spacing between the measurement vessel and the piezoelectric element was set at 2.5 cm. As the high speed preamplifier for amplifying the photoacoustic signal intensity, an ultrasonic wave preamplifier (Model 5682, manufactured by Olympus Corp.) having an amplification factor of +30 dB was used. The amplified signal was input into a digital oscilloscope (DPO4104, manufactured by Tektronix Inc.). From outside the glass vessel, the polystyrene cuvette was irradiated with the pulse laser light. A fraction of the scattered light occurring accordingly was detected with a photodiode and input into the digital oscilloscope as a trigger signal. The digital oscilloscope was set at a 32 run average display mode, and the measurement of the photoacoustic signal intensity averaged over 32 runs of the laser pulse irradiation.

(Verification of Accumulation at Tumor Sites)

In the verification of the accumulation at tumor sites, female outbred BALB/c Slc-nu/nu mice (6 weeks of age at the time of purchase) (Japan SLC, Inc.) were used. For one week before the mice were made to bear a tumor, a standard diet and a standard bed were used, and the mice were acclimated in an environment allowing the mice to freely take diet and drinking water. N87 (human stomach cancer cell), Suit-2 (human pancreatic cancer cell), colon (mouse colon cancer cell), and CT26-HER2 cell carcinoma cell prepared by introducing into colon 26 epidermal growth factor receptor 2 (Human Epidermal Growth Factor Receptor 2, hereinafter abbreviated as HER2 in some cases) gene, a member of EGFR family, were subcutaneously injected into the mice. Before the start of the experiment, all the tumors were established, and the weights of the mice were 17 to 22 g. In the tails of the mice made to bear tumors, 100 μL of a particle dispersion (13 nmol or 104 nmol as a dye) was intravenously injected.

Next, the mice to which the particle dispersion was administered were subjected to euthanasia at 24 hours after the administration, and N87 tumor, Suit-2 tumor, colon 26 tumor and CT26-HER2 tumor were respectively extracted. Each of the tumor tissues was transferred to a plastic tube, a 1% Triton-X100 aqueous solution was added in an amount of 1.25 times the weight of the tumor tissue and the resulting mixture was homogenized. Next, to the homogenized mixture, tetrahydrofuran (THF) was added in an amount of 20.25 times the weight of the tumor tissue. The amount of the dye in the tumor tissue was quantitatively determined by measuring the fluorescence intensity of the homogenate solution as it was in the plastic tube by using the IVIS (registered trademark) Imaging System 200 Series (Xenogen Corp.).

### (Silicon 2,3-Naphthalocyanine Bis(trihexylsilyloxide)-Containing Nanoparticles)

### Example 1

### Synthesis of Nanoparticle (NP1)

Silicon 2,3-naphthalocyanine bis(trihexylsilyloxide) (hereinafter, abbreviated as compound 1, in some cases) (4.4 mg, manufactured by Sigma Aldrich Japan K.K.) was dissolved in 1.6 mL of chloroform, to prepare a dye chloroform solution.

Next, an aqueous solution (20 mL) dissolving Tween 20 (180 mg, manufactured by Tokyo Chemical Industry Co., Ltd., abbreviated as Tw20 in some cases) was stirred at room temperature for 20 minutes or more, and then the dye chloroform solution was dropwise mixed with the aqueous solution, and the resulting mixed solution was stirred for 30 minutes. Subsequently, the mixed solution was treated for 90 seconds with an ultrasonic disperser to prepare an O/W type emulsion.

Next, the emulsion was stirred under a heated condition (40° C.) to remove chloroform from the dispersoid. Then, the emulsion was centrifuged at 20000 g at 4° C. for 45 minutes, and the precipitated fraction was collected to yield a nanoparticle (NP1). Examples of the centrifugal separator include a high-speed refrigerated micro centrifuge (MX-300, manufactured by Tomy Seiko Co., Ltd.).

### Example 2

### Synthesis of Nanoparticle (NP2)

A nanoparticle (NP2) was obtained in the same manner as in Example 1 except that the aqueous solution dissolving Tween 20 was altered to an aqueous solution dissolving Tween 20 (180 mg, manufactured by Tokyo Chemical Industry Co., Ltd.) and SUNBRIGHT (registered trademark) DSPE-020PA (11 mg, manufactured by NOF Corp., hereinafter, abbreviated as DA in some cases).

### Example 3

### Synthesis of Nanoparticles (NP3)

A nanoparticle (NP3) was obtained in the same manner as for the foregoing NP2 except that DA was altered to SUNBRIGHT (registered trademark) DSPE-020-CN (11 mg, manufactured by NOF Corp., hereinafter abbreviated as DO2k in some cases), a phospholipid.

### Example 4

### Synthesis of Nanoparticle (NP4)

A nanoparticle (NP4) was obtained in the same manner as for the foregoing NP2 except that DA was altered to SUNBRIGHT (registered trademark) DSPE-050-CN (11 mg, manufactured by NOF Corp., hereinafter abbreviated as DO5k in some cases), a phospholipid.

### Example 5

### Synthesis of Nanoparticle (NP5)

A nanoparticle (NP5) was obtained in the same manner as for the foregoing NP2 except that DA was altered to Methoxyl PEG DSPE, Mw 10000 (11 mg, manufactured by Nanocs Inc., hereinafter abbreviated as DO10k in some cases), a phospholipid.

### Example 6

### Synthesis of Nanoparticle (NP6)

A nanoparticle (NP6) was obtained in the same manner as for the foregoing NP2 except that DA was altered to Methoxyl PEG DSPE, Mw 20000 (11 mg, manufactured by Nanocs Inc., hereinafter abbreviated as D020k in some cases), a phospholipid.

### Example 7

### Synthesis of Nanoparticle (NP7)

A nanoparticle (NP7) was prepared in the same manner as in Example 1 except that the method for collecting the particles was altered. In the collection method, the emulsion was centrifuged at 20000 g at 4° C. for minutes, and the supernatant fraction was collected. Then, the supernatant fraction was ultracentrifuged at 72100 g at 4° C. for 15 minutes, and the supernatant fraction was collected. Subsequently, the supernatant fraction was ultracentrifuged at 451000 g at 4° C. for 15 minutes, and the precipitated fraction was collected. Examples of the ultracentrifugal separator include himac CS150GXL (manufactured by Hitachi Koki Co., Ltd.).

### Example 8

### Synthesis of Nanoparticle (NP8)

A nanoparticle (NP8) was obtained in the same manner as in Example 7 except that the aqueous solution dissolving Tween 20 was altered to an aqueous solution dissolving Tween 20 (180 mg, manufactured by Tokyo Chemical Industry Co., Ltd.) and DA (11 mg, manufactured by NOF Corp.).

Table 1 shows the particle size, entrapment efficiency (EE) and the accumulation at tumor sites of each of the nanoparticles NP1, NP2, NP3, NP4, NP5, NP6, NP7 and NP8. The EE was obtained by dividing the collected amount of the compound 1 contained in the collected nanoparticles by the total amount of the compound 1 and expressed in terms of percentage.

TABLE 1

| | | | | Accumulation | |
|---|---|---|---|---|---|
| | | Particle | | at tumor sites | |
| | Surfactant | size | EE | (% ID/g) | |
| Nanoparticle | species | (nm) | (%) | N87 | Suit-2 |
| NP1 | Tw20 | 105 | 36 | 0.1 | — |
| NP2 | Tw20 + DA | 85 | 17 | 1.2 | 0.3 |
| NP3 | Tw20 + DO2k | 118 | 14 | 0.5 | — |
| NP4 | Tw20 + DO5k | 110 | 12 | 0.2 | — |
| NP5 | Tw20 + DO10k | 107 | 19 | 1.2 | — |
| NP6 | Tw20 + DO20k | 105 | 23 | 1.1 | — |
| NP7 | Tw20 | 10 | 7 | 1.1 | — |
| NP8 | Tw20 + DA | 19 | — | 6.7 | 2.8 |

### Example 9

### Synthesis of Nanoparticle (NP9)

The compound 1 (8.8 mg, manufactured by Sigma Aldrich Japan K.K.) was dissolved in 16 mL of chloroform to prepare a dye chloroform solution.

Next, an aqueous solution (200 mL) dissolving Polysorbate 20 (1800 mg, manufactured by Wako Pure Chemical Industries, Ltd.) was stirred at room temperature for 20 minutes or more, then the dye chloroform solution was dropwise mixed with the aqueous solution, and the resulting mixed solution was stirred for 30 minutes. Subsequently, the mixed solution was treated with an ultrasonic disperser for 90 seconds to prepare an O/W type emulsion.

Next, the emulsion was stirred under a heated condition (40° C.) to remove chloroform from the dispersoid. Subsequently, the emulsion was purified by using an ultrafiltration membrane (Omega ultrafiltration membrane disc filter, 300K, manufactured by Nihon Pall Corp.), and then, concentrated by using an ultrafiltration membrane (Ultracel 50K, manufactured by Merck KGaA) to yield a nanoparticle (NP9).

### Example 10

### Synthesis of Nanoparticle (NP10)

A nanoparticle 10 (hereinafter, abbreviated as NP10) was obtained in the same manner as for the foregoing NP9 except that Polysorbate 20 was altered to Polysorbate 80 (HX2) (420 mg, manufactured by NOF Corp.).

Table 2 shows the particle size, the small particle ratio and the accumulation at tumor sites for each of the nanoparticles NP9 and NP10. The accumulation at tumor sites of NP10 was evaluated according to the foregoing method using the dye in an amount of 13 nmol and/or 104 nmol. The derivation of the small particle ratio was performed as follows: the solution before the purification was centrifuged at 100000 g at 4° C. for 15 minutes, the supernatant fraction was collected, the collected amount of the compound 1 contained in the collected supernatant fraction was divided by the total amount of the compound 1, and the resultant quotient was expressed in terms of percentage.

TABLE 2

| | | | | | Accumulation | |
|---|---|---|---|---|---|---|
| | | | | Dye | at tumor | |
| | | | Small | administra | sites | |
| | | Particle | particle | -tion | (% ID/g) | |
| Nano- | Surfactant | size | ratio | amount | | CT26 + |
| particle | species | (nm) | (%) | (nmol) | CT26 | HER2 |
| NP9 | Polysorbate | 9 | 83 | 13 | 27.2 | 17.6 |
| | 20 | | | | | |
| NP10 | Polysorbate | 11 | 67 | 13 | 26.9 | 9.7 |
| | 80 | | | 104 | 24.6 | 12.6 |

As can be verified from the results in Table 2, in the case of NP10, even when the administration amount thereof was altered, similar accumulations at tumor sites were obtained.

### Example 11

### Synthesis of Nanoparticle (NP11)

The compound 1 (0.88 mg, manufactured by Sigma Aldrich Japan K.K.) was dissolved in 1.6 mL of chloroform to prepare a dye chloroform solution.

Next, an aqueous solution (20 mL) dissolving DO2k (180 mg, manufactured by NOF Corp.), a phospholipid was stirred at room temperature for 20 minutes or more, then the dye chloroform solution was dropwise mixed with the aqueous solution, and the resulting mixed solution was stirred for 30 minutes. Subsequently, the mixed solution was treated for 90 seconds with an ultrasonic disperser to prepare an O/W type emulsion.

Next, the emulsion was stirred under a heated condition (40° C.) to remove chloroform from the dispersoid. Then, the emulsion was centrifuged at 100000 g at 4° C. for 15 minutes, and the supernatant fraction was collected to yield a nanoparticle (NP11).

### Example 12

### Synthesis of Nanoparticle (NP12)

A nanoparticle (NP12) was obtained in the same manner as for the foregoing NP11 except that DO2k was altered to DO5k (180 mg, manufactured by NOF Corp.), a phospholipid.

### Example 13

### Synthesis of Nanoparticle (NP13)

A nanoparticle (NP13) was obtained in the same manner as for the foregoing NP11 except that DO2k was altered to DA (180 mg, manufactured by NOF Corp.), a phospholipid.

### Example 14

### Synthesis of Nanoparticle (NP14)

A nanoparticle (NP14) was obtained in the same manner as for the foregoing NP11 except that DO2k was altered to SUNBRIGHT (registered trademark) DSPE-020MA (180 mg, manufactured by NOF Corp., hereinafter abbreviated as DM in some cases), a phospholipid.

### Example 15

### Synthesis of Nanoparticle (NP15)

A nanoparticle (NP15) was obtained in the same manner as for the foregoing NP11 except that DO2k was altered to Pluronic F68 (180 mg, manufactured by Sigma Aldrich Japan K.K., hereinafter abbreviated as F68 in some cases).

Table 3 shows the particle size, the small particle ratio and the accumulation at tumor sites for each of the nanoparticles NP11, NP12, NP13, NP14 and NP15.

TABLE 3

| | | Particle | Small | | | |
|---|---|---|---|---|---|---|
| | | size after | particle | Accumulation at | | |
| Nano- | Surfactant | preparation | ratio | tumor sites | | |
| particle | species | (nm) | (%) | N87 | Suit-2 | CT26 |
| NP11 | DO2k | 12 | 64 | 7.5 | 4.9 | — |
| NP12 | DO5k | 19 | 66 | 5.1 | 2.5 | — |
| NP13 | DA | 15 | 31 | 4.9 | 4.2 | — |
| NP14 | DM | 26 | 63 | 3.3 | — | 8.3 |
| NP15 | F68 | 17 | 19 | — | — | — |

### Example 16

### Preparation of Single-Chain Antibody hu4D5-8scFv

Based on the gene sequence (hu4D5-8) in the variable region of a HER2-binding IgG, a gene hu4D5-8scFv encoding a single-chain antibody (scFv) was prepared. First, a cDNA including the VL and VH genes of the hu4D5-8 linked via a cDNA encoding a peptide (GGGGS)₃ was prepared. A restriction enzyme NcoI- was introduced to the 5′-terminal, and a recognition site of a restriction enzyme NotI was introduced to the 3′-terminal. The base sequence is shown below.

Sequence No. 1:
5′-CCATGGATATCCAGATGACCCAGTCCCCGAGCTCCCTGTCCGCC
TCTGTGGGCGATAGGGTCACCATCACCTGCCGTGCCAGTCAGGATGT
GAATACTGCTGTAGCCTGGTATCAACAGAAACCAGGAAAAGCTCCGA
AACTACTGATTTACTCGGCATCCTTCCTCTACTCTGGAGTCCCTTCT
CGCTTCTCTGGATCCAGATCTGGGACGGATTTCACTCTGACCATCAG
CAGTCTGCAGCCGGAAGACTTCGCAACTTATTACTGTCAGCAACATT
ATACTACTCCTCCCACGTTCGGACAGGGTACCAAGGTGGAGATCAAA
GGCGGTGGTGGCAGCGGTGGCGGTGGCAGCGGCGGTGGCGGTAGCGA
GGTTCAGCTGGTGGAGTCTGGCGGTGGCCTGGTGCAGCCAGGGGGCT
CACTCCGTTTGTCCTGTGCAGCTTCTGGCTTCAACATTAAAGACACC
TATATACACTGGGTGCGTCAGGCCCCGGGTAAGGGCCTGGAATGGGT
TGCAAGGATTTATCCTACGAATGGTTATACTAGATATGCCGATAGCG
TCAAGGGCCGTTTCACTATAAGCGCAGACACATCCAAAAACACAGCC
TACCTGCAGATGAACAGCCTGCGTGCTGAGGACACTGCCGTCTATTA
TTGTTCTAGATGGGGAGGGGACGGCTTCTATGCTATGGACTACTGGG
GTCAAGGAACCCTGGTCACCGTCTCCTCGGCGGCCGC-3′

(The recognition site of the restriction enzyme is underlined.)

The gene fragment hu4D5-8scFv was inserted downstream of the T7/lac promoter of a plasmid pET-22b(+) (Novagen, Inc.). Specifically, the cDNA is ligated to the pET-22b(+) digested with the restriction enzyme NcoI- and the restriction enzyme NotI.

The expression plasmid was transformed into an Escherichia coli (Escherichia coli BL21(DE3)) to yield a strain for expression. The obtained strain was precultured in 4 ml of a LB-Amp medium overnight, and then the total volume was added to a 250 ml of a 2xYT culture medium, and it was shake-cultured at 28° C. at 120 rpm for 8 hours. Then, Isopropyl-beta-D(−)-thiogalactopyranoside (IPTG) was added to the culture medium in a final concentration of 1 mM, and the strain was cultured at 28° C. overnight. The cultured Escherichia coli was centrifuged at 8000×g for 30 minutes at 4° C., and the supernatant culture medium was collected. To the obtained culture medium, ammonium sulfate was added in an amount of 60% of the obtained culture medium, and protein was precipitated by salting out. The solution subjected to the salting out operation was allowed to stand still overnight at 4° C., and then centrifuged at 8000×g for 30 minutes at 4° C. to collect the precipitate. The obtained precipitate was dissolved in a buffer 20 mM Tris•HCl/500 mM NaCl and was dialyzed to 1 l of the same buffer. The protein solution after the dialysis was added to a column packed with His•Bind (registered trademark) Resin (Novagen, Inc.), and purified by metal-chelate affinity chromatography using Ni ion. The purified hu4D5-8scFv exhibited a single band with the reducing SDS-PAGE, and the molecular weight was verified to be about 28 kDa. The amino acid sequence of the prepared antibody is shown below. Hereinafter, the hu4D5-8scFv is abbreviated as scFv.

Sequence No. 2:
DIQMTQSPSSLSASVGDRVTITCRASQDVNTAVAWYQQKPGKAPKLL
IYSASFLYSGVPSRFSGSRSGTDFTLTISSLQPEDFATYYCQQHYTT
PPTFGQGTKVEIKGGGGSGGGGSGGGGSEVQLVESGGGLVQPGGSLR
LSCAASGFNIKDTYIHWVRQAPGKGLEWVARIYPTNGYTRYADSVKG
RFTISADTSKNTAYLQMNSLRAEDTAVYYCSRWGGDGFYAMDYWGQG
TLVTVSSAAALEHHHHHHGGC

### Example 17

### Modification of NP2 Surface with scFv

The scFv prepared in Example 16 was subjected to the substitution of the buffer with a 5 mM EDTA-containing phosphoric acid buffer (2.68 mM KCl/137 mM NaCl/1.47 mM KH₂PO₄/1 mM Na₂HPO₄/5 mM EDTA, pH 7.4), and was subjected to a reduction treatment with a 10-fold molar amount of tris(2-carboxyethyl)phosphine hydrochloride (TCEP) at 25° C. for about 2 hours.

Via the primary amino groups present on the surface of the NP2, the modification with the scFv was performed. First, 0.1 mg (233 nmol) of succinimidyl-[(N-maleimidopropionamido)-diethyleneglycol]ester (SM(PEG)₂, Thermo Scientific, Inc.) was dissolved in 0.5 ml of a aqueous dispersion of NP2 (NP concentration: 1.1E-08 M). Next, 0.056 ml of a boric acid buffer (pH 8.5) was added to the aqueous dispersion. The particle suspension was stirred at room temperature overnight, and then the NP2 having the maleimide group introduced thereto (hereinafter, abbreviated as the maleimidized NP2) and the unreacted SM(PEG)₂ were separated by using a PD-10 desalting column (manufactured by GE Healthcare Bioscience Ltd.) and water as the eluent, to yield an aqueous solution of the maleimidized NP2. To the aqueous solution, a 1M 2-[4-(2-Hydroxyethyl)-1-piperazinyl]ethanesulfonic acid (HEPES) solution was added so as for the final concentration to be 20 mM to yield a HEPES solution of the maleimidized NP2.

The scFv subjected to a reduction treatment was added to the HEPES solution of the maleimidized NP2 and was allowed to react with the maleimidized NP2 at 4° C. overnight. The reaction was performed with a reaction molar ratio (scFv/maleimidized NP2) at the time of preparation of 1000. Here, the “preparation” means the addition to the reaction system, and the “reaction molar ratio at the time of preparation” means the molar concentration ratio between the scFv and the maleimidized NP20 added to the reaction system. After the reaction, to the solution, 4.2 nmol of a polyethylene glycol having a terminal thiol group (molecular weight: 1000, PLS-606, manufactured by Creative PEGWorks, Inc.) was added and stirred at room temperature for 30 minutes. Next, from the solution, the scFv unbound to the maleimidized NP2 was removed by an ultrafiltration using Amicon Ultra-4 (Nihon Millipore K.K.) having a pore size of 100 kDa to yield scFv-modified nanoparticles (scFv-NP2).

### Example 18

### Modification of NP8 Surface with scFv

The reduction treatment of the scFv was performed in the same manner as in Example 17.

Via the primary amino groups present on the surface of the NP8, the modification with the scFv was performed. First, 0.25 mg (582.5 nmol) of succinimidyl-[(N-maleimidopropionamido)-diethyleneglycol]ester (SM(PEG)2, Thermo Scientific, Inc.) was dissolved in 1.0 ml of an aqueous dispersion of NP8 (NP concentration: 5.7E-06 M). Next, 0.111 ml of a boric acid buffer (pH 8.5) was added to the aqueous dispersion. The particle suspension was stirred at room temperature overnight, and then the NP8 having the maleimide group introduced thereto (hereinafter, abbreviated as the maleimidized NP8) and the unreacted SM(PEG)₂ were separated by using a PD-10 desalting column (manufactured by GE Healthcare Bioscience Ltd.) and water as the eluent, to yield an aqueous solution of the maleimidized NP8. To the aqueous solution, a 1M 2-[4-(2-Hydroxyethyl)-1-piperazinyl]ethanesulfonic acid (HEPES) solution was added so as for the final concentration to be 20 mM to yield a HEPES solution of the maleimidized NP8.

The scFv subjected to a reduction treatment was added to the HEPES solution of the maleimidized NP8 and was allowed to react with the maleimidized NP8 at 4° C. overnight. The reaction was performed with a reaction molar ratio (scFv/maleimidized NP8) at the time of preparation of 10. Here, the “preparation” means the addition to the reaction system, and the “reaction molar ratio at the time of preparation” means the molar concentration ratio between the scFv and the maleimidized NP20 added to the reaction system. After the reaction, to the solution, 42 nmol of a polyethylene glycol having a terminal thiol group (molecular weight: 1000, PLS-606, manufactured by Creative PEGWorks, Inc.) was added and stirred at room temperature for 30 minutes. Next, from the solution, the scFv unbound to the maleimidized NP8 was removed by an ultrafiltration using Amicon Ultra-4 (Nihon Millipore K.K.) having a pore size of 100 kDa to yield scFv-modified nanoparticles (scFv-NP8).

Table 4 shows the particle size, the modification amount of scFv per particle and the accumulation at tumor sites for each of the nanoparticles scFv-NP2, NP2, scFv-NP8 and NP8. The modification amount of scFv per particle was derived by using the BCA (bicinchoninic acid) method.

TABLE 4

| | | Number of | Accumulation | |
|---|---|---|---|---|
| | Particle | scFv (per | at tumor sites | |
| Nanoparticle | size (nm) | particle) | N87 | Suit-2 |
| scFv-NP2 | 79 | 640 | 3.6 | 1.3 |
| NP2 | 85 | 0 | 1.2 | 0.3 |
| scFv-NP8 | 28 | 3.2 | 10.0 | 0.6 |
| NP8 | 19 | 0 | 6.7 | 2.8 |

As can be seen from the results shown in Table 4, the particles modified with scFv were higher in the accumulation at tumor sites for N87, a HER2 positive cell. As has also been verified from the results shown in Table 4, the particles modified with scFv were higher by a factor of 3 or more in the accumulation at tumor sites for a HER2 positive cell N87 than for a HER2 negative cell Suit-2, and thus, the scFv-NP2 and the scFv-NP8, both having the HER2 binding function, were selectively accumulated on N87. Therefore, the nanoparticles prepared in present Example are regarded as suitable as a contrast agent for the photoacoustic imaging of tumor.

### Example 19

### Synthesis of Nanoparticle (NP101)

Preparation of Organic Solvent Solution:

The compound 1 (0.88 mg, manufactured by Sigma Aldrich Japan K.K.) was dissolved in 4.0 mL of tetrahydrofuran (hereinafter, abbreviated as THF in some cases) to prepare a dye solution. The obtained solution was a green, transparent solution.

Preparation of a Dispersion of a Dispersion Stabilizer:

An aqueous solution (20 mL) dissolving Tween 20 (180 mg, manufactured by Tokyo Chemical Industry Co., Ltd.) was stirred at room temperature for 20 minutes or more to prepare a dispersion of a dispersion stabilizer.

Preparation of Particle Dispersion:

While the dispersion of the dispersion stabilizer was being stirred with a magnetic stirrer, the dye THF solution was dropwise added to the dispersion of the dispersion stabilizer, and the stirring was continued for minutes from the start of the dropwise addition to prepare a particle dispersion.

Distillation Off of Solvent:

The particle dispersion was put in a water bath (BM100, manufactured by Yamato Scientific Co., Ltd.) set at 40° C. and stirred at 800 rpm for 2 hours. Then, the heater of the water bath was turned off, and the particle dispersion was continuously stirred as it was at room temperature for 16 hours.

Purification of Particles:

The nanoparticle dispersion obtained in the forgoing steps was centrifuged with an ultracentrifugal separator (himac CS150GXL, manufactured by Hitachi Koki Co., Ltd.) at 4° C. at 100,000 g for 15 minutes, and 600 μL of the supernatant fraction was collected.

The collected liquid was sequentially filtered with 5.0 μm, 1.2 μm, 0.8 μm, 0.45 μm and 0.22 μm filters to yield a nanoparticle (NP101). The particle size and the EE of NP101 were found to be 14.7 nm and 37%, respectively.

### Example 20

### Synthesis of Nanoparticle (NP102)

A nanoparticle (NP102) was obtained in the same manner as for the foregoing NP101 except that Tween 20 was altered to dextran 40 (180 mg, manufactured by Tokyo Chemical Industry Co., Ltd.) and the step of purifying the particles was altered from the ultracentrifugal separation to the filtration with a 0.22 μm filter. The particle size of NP102 was found to be 13.8 nm.

### Example 21

### Verification of Tumor Imaging Performance

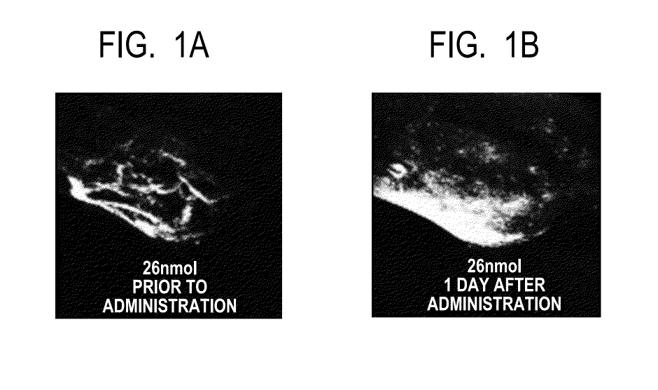

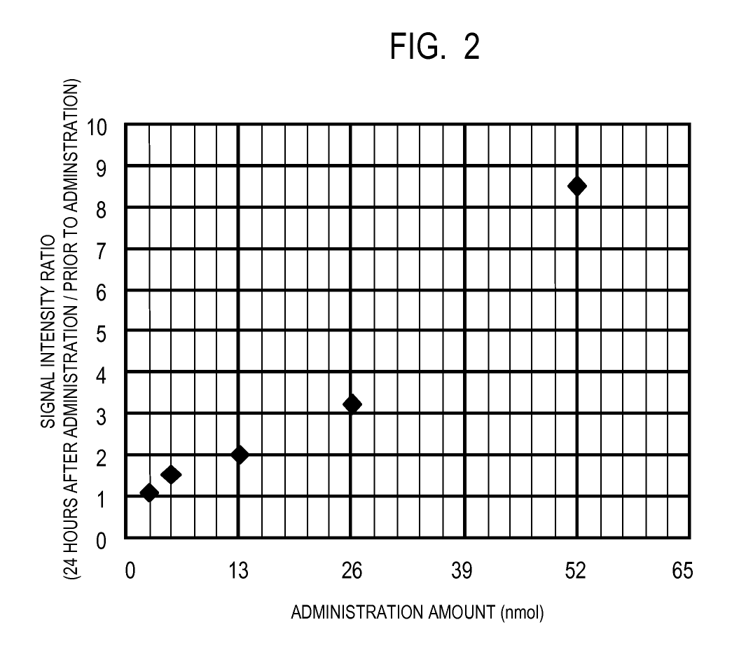

The photoacoustic signal of the tumor site of the foregoing colon 26-bearing mouse prior to administration was measured by using the Nexus 128 (manufactured by Endra Inc.). Next, the NP9 particle dispersion was administered from the tail vein, in the dye amounts of 2.6, 5.2, 13, 26 and 52 nmol, and photoacoustic signals from the tumor sites at 24 hours after the administration were measured in the same manner as described above. FIG. 1A is the photoacoustic image of a tumor site of a tumor-bearing mouse, prior to the administration of NP9 to the mouse. FIG. 1B is the photoacoustic image of the tumor site of the tumor-bearing mouse, after the passage of 24 hours from the administration of 26 nmol of NP9 as a dye amount to the tumor-bearing mouse. FIG. 2 shows the ratio of the photoacoustic signal intensity at 24 hours after the administration to the photoacoustic signal intensity prior to the administration for each of the dye administration amounts. As has been verified from FIGS. 1A and 1B, as compared to prior to the administration, the signal intensity of the tumor site at 24 hours after the administration is improved. As has also been verified from FIG. 2, with the increase of the amount of the administered dye, the signal intensity ratio is also increased. From these verifications, NP9 has been shown to enable imaging of the tumor and the effectiveness of NP9 as the tumor contrast agent for photoacoustic imaging method has been shown.

### Comparative Example 1

### Synthesis of Nanoparticle (NPA1)

A nanoparticle (NPA1) was obtained by using the compound 1 and Tween 80 (manufactured by Sigma Inc.), according to NPL 1. The particle size and the recovery percentage of NPA1 were found to be 142 nm and 5%, respectively. The recovery percentage was derived by dividing the collected amount of the compound 1 by the total amount of the compound 1.

### Comparative Example 2

### Synthesis of Polymer Nanoparticle (PNP1)

The compound 1 (0.88 mg, manufactured by Sigma Aldrich Japan K.K.) and poly(lactic-co-glycolic acid) (PLGA) (5 mg, composition ratio of lactic acid to glycolic acid: 50:50, average molecular weight: 20000, manufactured by Wako Pure Chemical Industries, Ltd.) were dissolved in 1.6 mL of chloroform to prepare a dye chloroform solution.

Next, an aqueous solution (20 mL) dissolving Tween (180 mg, manufactured by Tokyo Chemical Industry Co., Ltd.) was stirred at room temperature for 20 minutes or more, then the dye chloroform solution was dropwise mixed with the aqueous solution, and the mixed solution was stirred for 30 minutes. Subsequently, the mixed solution was treated with an ultrasonic disperser for 90 seconds to prepare an O/W type emulsion.

Next, the emulsion was stirred under a heated condition (40° C.) to remove chloroform from the dispersoid, and then from the emulsion, the excessive surfactant was removed by ultrafiltration or centrifugal separation operation to yield a polymer nanoparticle (PNP1) with the surface thereof protected with Tween 20 and the compound 1 contained in PLGA. The particle size and the EE of PNP1 were found to be 107 nm and 42%, respectively.

Table 5 shows the photoacoustic signal (PA signal) intensity per dye molecule, the PA signal per particle and the proportion of the dye in relation to the other component of the particle exclusive of the surfactant for each of the nanoparticles NP1, NP2, NP3, NP4, NP7, NP8, NP9, NP10, NPA1 and PNP1. The proportion of the dye in relation to the other component of the particle exclusive of the surfactant was derived from the solid weight of each of the constituent materials in the sample obtained by the NMR measurement, the absorbance measurement and the freeze dried weight measurement. The NMR measurement was performed by using an NMR spectrometer (AVANCE 500 manufactured by Bruker Corp., resonance frequency: 500 MHz, measurement nuclear species: 1H, measurement temperature: room temperature, solvent: heavy chloroform).

TABLE 5

| | | | PA signal | Proportion of |
|---|---|---|---|---|
| | | | per | dye in relation |
| | | | particle in | to the other |
| | | PA signal | terms of | component of |
| | | per dye | 100 nm | the particle |
| | Surfactant | molecule | particle | exclusive of |
| Nanoparticle | species | (V/J/dyeM) | (V/J/M) | surfactant (%) |
| NP1 | Tw20 | 1.5E+07 | 1.6E+12 | 96 |
| NP2 | Tw20 + DA | 1.8E+07 | 8.9E+11 | — |
| NP3 | Tw20 + DO2k | 1.1E+07 | 2.2E+12 | — |
| NP4 | Tw20 + DO5k | 4.2E+06 | 9.0E+11 | — |
| NP7 | Tw20 | 1.2E+07 | 3.0E+10 | — |
| NP8 | Tw20 + DA | 1.7E+07 | 6.5E+10 | — |
| NP9 | Polysorbate | 9.2E+06 | 2.4E+10 | — |
| | 20 | | | |
| NP10 | Polysorbate | 1.1E+07 | 3.3E+10 | — |
| | 80 | | | |
| NPA1 | Tween80 | 4.2E+06 | — | — |
| | (Comparative | | | |
| | Example 1) | | | |
| PNP1 | PNP | 1.3E+07 | 9.9E+10 | 45 |
| | (Comparative | | | |
| | Example 2) | | | |

As can be seen from the results shown in Table 5, as compared to PNP1, the nanoparticles of the present invention NP1, NP2, NP3, NP4, NP7, NP8, NP9 and NP10 can increase the proportion of the dye without weakening the signal intensity (light absorptivity) per dye molecule. Therefore, when the nanoparticle of the present invention is used as a contrast agent, the absorption efficiency of the irradiation energy is increased and high intensity signals can be obtained.

### Example 22

### Derivation of Photoacoustic Signal Intensity from Tumor

The photoacoustic signal from the tumor was derived based on the following formula.

Photoacoustic signal from tumor=accumulated amount (nmol/g) at tumor sites at 24 hours after administration×photoacoustic signal (V/J/dye M) per dye molecule in particles

The accumulated amount (nmol/g) at tumor sites at 24 hours after the administration of NPA1 was derived from the accumulated amount at tumor sites described in NPL 1 with the molecular weight of isoBOSINC set at 1564.44.

Table 6 shows, for each of NP9 and NPA1, the accumulated amount (nmol/g) at tumor sites, the photoacoustic signal (V/J/dye M) per dye molecule in particles and the intensity of the photoacoustic signal from the tumor at 24 hours after the administration of the concerned nanoparticle. As can be seen from the results shown in Table 6, as compared to NPA1, the nanoparticle NP9 of the present invention is higher both in the accumulation at tumor sites and in the photoacoustic signal intensity per particle. Therefore, NP9 can increase the intensity of the photoacoustic signal from the tumor, and is effective as the contrast agent of the photoacoustic imaging method.

TABLE 6

| | Accumulated | Photoacoustic | |
|---|---|---|---|
| | amount at | signal per dye | Photoacoustic |
| | tumor sites | molecule | signal from |
| Nanoparticle | (nmol/g) | (V/J/dyeM) | tumor |
| NP9 | 3.5 | 9.2E+06 | 3.2E+07 |
| NPA1 | 1.1 | 4.2E+06 | 4.6E+06 |

Preferred embodiments of the present invention will now be described in detail in accordance with the accompanying drawings.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Applications No. 2012-038036, filed Feb. 23, 2012 and No. 2012-263003, filed Nov. 30, 2012, which are hereby incorporated by reference herein in their entirety.

What is claimed is:

1. A nanoparticle comprising at least a silicon naphthalocyanine or a derivative thereof and a surfactant, wherein the proportion of the silicon naphthalocyanine or the derivative thereof in relation to the other components of the particle exclusive of the surfactant is 70% or more by weight.

2. The nanoparticle according to claim 1, wherein the silicon naphthalocyanine or the derivative thereof is represented by the chemical formula 1: 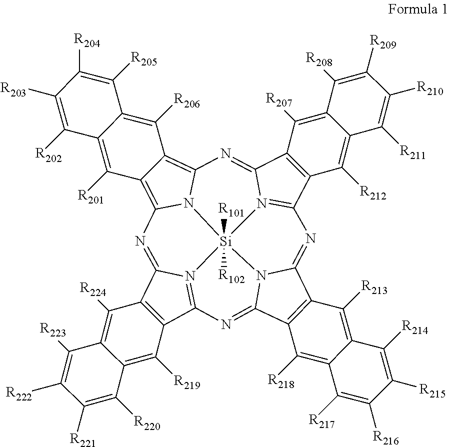
(in the formula, R₂₀₁, R₂₀₂, R₂₀₃, R₂₀₄, R₂₀₅, R₂₀₆, R₂₀₇, R₂₀₈, R₂₀₉, R₂₁₀, R₂₁₁, R₂₁₂, R₂₁₃, R₂₁₄, R₂₁₅, R₂₁₆, R₂₁₇, R₂₁₈, R₂₁₉, R₂₂₀, R₂₂₁, R₂₂₂, R₂₂₃ and R₂₂₄ may each be the same or different, and each represent a hydrogen atom, a halogen atom, an acetoxy group, an amino group, a nitro group, a cyano group or an alkyl group having 1 to 18 carbon atoms or aromatic group, unsubstituted or substituted with one or a plurality of the functional groups selected from a halogen atom, an acetoxy group, an amino group, a nitro group, a cyano group and an alkyl group having 1 to 18 carbon atoms;
additionally, R₁₀₁ and R₁₀₂ may each be the same or different, and each represent —OH, —OR₁₁, —OCOR₁₂, —OSi(—R₁₃)(—R₁₄)(—R₁₅), a halogen atom, an acetoxy group, an amino group, a nitro group, a cyano group or an alkyl group having 1 to 18 carbon atoms or aromatic group, unsubstituted or substituted with one or a plurality of the functional groups selected from a halogen atom, an acetoxy group, an amino group, a nitro group, a cyano group and an alkyl group having 1 to 18 carbon atoms;
here, R₁₁, R₁₂, R₁₃, R₁₄ and R₁₅ may each be the same or different, each represent a group unsubstituted or substituted with one or a plurality of the functional groups selected from a halogen atom, an acetoxy group, an amino group, a nitro group, a cyano group and an alkyl group having 1 to 18 carbon atoms).

3. The nanoparticle according to claim 1, wherein the silicon naphthalocyanine or the derivative thereof is any one of silicon 2,3-naphthalocyanine dioctyloxide, silicon 2,3-naphthalocyanine dichloride, bis(di-isobutyl octadecylsiloxy)silicon 2,3-naphthalocyanine and silicon 2,3-naphthalocyanine bis(trihexylsilyloxide).

4. The nanoparticle according to claim 1, wherein the average particle size thereof is 5 nm or more and 200 nm or less.

5. The nanoparticle according to claim 1, wherein the nanoparticle is used as a contrast agent.

6. The nanoparticle according to claim 1, wherein the nanoparticle is used as a contrast agent for a photoacoustic imaging method.

7. The nanoparticle according to claim 1, wherein the surfactant is represented by the chemical formula 2. 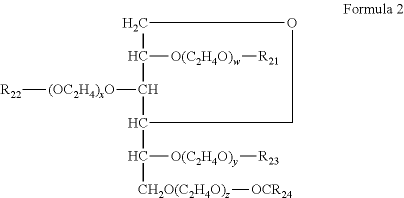
In the chemical formula 2, R₂₁ to R₂₄ are each independently selected from —H and —OCR′. The R′ is a saturated or unsaturated alkyl group having 1 to 18 carbon atoms. In the chemical formula 2, w, x, y and z are integers giving the sum of w, x, y and z to be 10 to 30.

8. The nanoparticle according to claim 1, further comprising a capture molecule to be specifically bound to a target site.
